(19) United States
(12) Patent Application Publication
(10) Pub. No.: US 2018/0207587 A1
(43) Pub. Date: Jul. 26, 2018

(54) DIALYZER INCLUDING IMPROVED INTERNAL FILTRATION AND METHOD OF MANUFACTURE THEREOF

(71) Applicant: B. BRAUN AVITUM AG, MELSUNGEN (DE)

(72) Inventor: Peter Mandry, Dresden (DE)

(21) Appl. No.: 15/877,729

(22) Filed: Jan. 23, 2018

(30) Foreign Application Priority Data
Jan. 24, 2017 (DE) 10 2017 101 307.5

(51) Int. Cl. B01D 61/28; A61M 1/34

(52) U.S. Cl. CPC B01D 61/28; A61M 2207/00; A61M 1/3413

(57) ABSTRACT

A dialyzer and a method of manufacture thereof, wherein the dialyzer includes a tubular dialyzer housing in the interior of which a plurality of capillaries each extending in the longitudinal direction of the dialyzer housing and being juxtaposed transversely to the longitudinal direction is arranged, with a filler having a volume-increasing property being arranged between the inner wall of the dialyzer housing and the capillaries.

### CROSS REFERENCE TO RELATED APPLICATIONS

This application claims priority to German application DE 10 2017 101 307.5 filed Jan. 24, 2017, the contents of such application being incorporated by reference herein.

### FIELD OF THE INVENTION

The present invention relates to a dialyzer for diffusive and convective matter transport for removing macromolecular particles.

### BACKGROUND OF THE INVENTION

It is the target of dialysis therapy, apart from detoxification of the blood, to remove excess water which due to renal insufficiency underlying the dialysis accumulates in the body from the latter. This is performed by so-called ultrafiltration during which liquid is removed from the blood via a dialyzer.

Conventional dialyzers usually include a tubular dialyzer housing having a longitudinal extension, with the interior of the dialyzer having a cross-section which typically does not vary or varies only insignificantly over the entire longitudinal extension. In the interior, capillary membranes (hollow fiber membranes) are provided to be arranged in parallel. The capillary membranes together form a portion of an extracorporeal blood circulation, while the exterior of the capillaries and the interior of the dialyzer housing form a portion of the circulation of the dialysis solution (dialysate). The two circulations circulate in opposite directions and are separated from each other by the semipermeable membranes of the capillaries. Through said semipermeable membranes, an exchange of both water and matter takes place. Especially, water and contaminants are removed from the patient's blood. Retention products increasing in diameter or in molecular weight are removed in dialyzers by diffusive processes through the membranes in a worse manner than smaller contaminants.

Different dialysis techniques used are, inter alia, hemodialysis, hemodiafiltration and high-flux dialysis.

Hemodialysis is carried out according to the principle of balancing the concentration of micromolecular substances of two fluids separated by a semipermeable membrane (osmosis). Separated from the filter membrane, on the side the blood including electrolytes such as potassium and phosphate as well as substances usually eliminated with the urine (e.g. urea, uric acid) is provided. On the other side of the membrane, a low-germ conditioned solution (dialysate) is provided the water of which was conditioned in online preparation by reverse osmosis and which contains no waste products and includes a portion of electrolytes orientated at the respective needs of the patient. The semipermeable filter membrane (dialysis membrane) between the blood and the dialysate has pores that allow small molecules such as water, electrolytes and substances usually eliminated with the urine to pass but withhold large molecules such as proteins and blood cells.

For hemodiafiltration the hemodialysis and a hemofiltration are employed in combination. This method is applied in particular in the case of chronical renal insufficiency and allows for both the removal of low-molecular as well as medium-molecular substances with a controlled replacement of the ultrafiltrate by physiological electrolyte solution (diluate). The replacement solution is added to the blood either before or after the dialyzer and is removed again in the dialyzer (ultrafiltration). In this way, higher transmembrane flow resulting in a more efficient removal of toxic substances can be produced.

Finally, the high-flux dialysis is understood to be hemodialysis having a high ultrafiltration coefficient (K_{UF}>10) which indicates the hourly ultrafiltration (Uf) in ml that is achieved per mmHg of transmembrane pressure (TMP).

FIG. 1 illustrates schematic representations of the functioning of the three afore-mentioned dialysis techniques by way of a diffusion direction and intensity indicated by arrows and the size thereof (left-hand diagrams) as well as a corresponding pressure profile (right-hand diagrams) along the tubular dialyzer housing, namely (a) for hemodialysis, (b) for hemodiafiltration and (c) for high-flux dialysis. As is evident by way of the diagrams in FIG. 1(a), in normal hemodialysis due to the low permeability of the membranes low ultrafiltration takes place between the blood (B) and the dialysate (D) despite a positive TMP gradient (low ultrafiltration coefficient). The diagrams shown in FIG. 1(b) indicate that during hemodiafiltration due to the use of membranes having high permeability with a similar TMP gradient a definitely higher ultrafiltration rate is achieved (high ultrafiltration coefficient). Finally, the diagrams in FIG. 1(c) illustrate that with high-flux dialysis due to volumetric control of high ultrafiltration rates through the dialysis apparatus a reversal of the pressure gradient along the dialyzer is resulting and a typical profile of the filtration/back-filtration is obtained in the dialyzer filter. Due to this pressure profile, the convection is maintained in the proximal area of the dialyzer (left side of the diagram) (convective matter transport), while in the distal area (right side of the diagram) back-filtration takes place by the change of diffusion direction related to the reversal of the pressure gradient, wherein the ultrafiltration can be significantly increased with said back-filtration. Thus, by the high-flux dialysis a convective component can be achieved so that internal filtration for removal of medium molecules is possible without re-infusion of a replacement solution.

The removal of so-called medium molecules (proteins/protein fragments) is to be considered a determining factor for differences in the survival rate of patients who are treated either with so-called low-flux dialyzers (K_{UF}<10) for mostly diffusive removal of micromolecular particles (molecules) with hardly any convective matter transport of proteins or with so-called high-flux dialyzers (K_{UF}>10) with diffusive and convective matter transport for removal of macromolecular particles (proteins up to 70 kDA). A high convective transport of several liters is obtained during a dialysis treatment by the fact that a high ultrafiltration rate is selected and the removed liquid quantity of the blood is replaced again with substitution liquid. The latter is obtained either from infusion solution or directly from the dialysate. Both options are linked with increased costs and efforts in terms of apparatuses.

### DESCRIPTION OF THE RELATED ART

From DE 102015100070 A1 a dialyzer housing is known which has an annular constriction at the inner side in the dialysate chamber. Said constriction entails increased pressure drop of the dialysate and thus increased back-filtration.

Furthermore, in Kidney International, Volume 54, Edition 3, September 1998, pages 979-985 illustrate a dialyzer having an improved convective performance by insertion of an O-ring around a membrane bundle.

Both aforementioned solutions for increasing the convective transport inside the dialyzer show the drawback that great efforts in terms of manufacture have to be made and thus the manufacturing costs are considerably increased. For example, the inexpensive mode of manufacturing the housing by injection molding permits no undercut in the interior and the thin wall thickness permits no constriction of the housing by heat and force from outside. An increase in the wall thickness would entail increased material requirements and longer cycle time.

Moreover, the introduction of the O-ring to the interior of the dialyzer is very difficult due to the space available. The fiber bundle (capillaries) is tightly wrapped by a film and is pushed or drawn into the dialyzer. Then the film is removed. In order to obtain high clearance (i.e. proper purifying effect) of the dialyzer, an as high packing density as possible is strived for in the dialyzer (namely, a maximum number of fibers in cross-section of the dialyzer). In this way, no space is left for an additional ring which might be inserted, in particular because it is very demanding already in conventional dialyzers to insert the bundle in a non-damaging manner.

### SUMMARY OF THE INVENTION

The object underlying the present invention inter alia is to influence the pressure gradient of a dialyzer with little additional manufacturing effort so that the internal filtration reaches the magnitude of hemodiafiltration with re-infusion.

This object is achieved by a dialyzer and a manufacturing method as defined in the claims.

Accordingly, to part of the wrapping film or to the fiber bundle (capillary bundle) itself a filler is applied which has volume-increasing properties and does not expand before it has been introduced to the housing. This facilitates insertion of the fiber bundle into the narrow area between the fiber bundle and the dialyzer housing. The development of the volume (activation of volume expansion) then can be triggered by different mechanisms depending on the filler. The expanded filler acts as a flow resistance and increases the differences in pressure between the dialysate and the blood.

Thus, according to aspects of the invention, simple manufacture of dialyzers for blood purification by internal filtration definitely improved with respect to conventional dialyzers is achieved. This results in better clearance of medium molecules and thus improved dialyzing action.

The suggested solution may also be used to increase the packing density of the fibers over the entire length of the dialyzer without the diameter of the bundle having to be enlarged when inserting the fibers. Here almost the entire film and, respectively, bundle is coated with the polymer.

Specific advantageous embodiments of the present invention are described in the subclaims.

### BRIEF DESCRIPTION OF THE DRAWINGS

The invention is best understood from the following detailed description when read in connection with the accompanying drawings. Included in the drawings are the following figures:

FIGS. 1(a), 1(b), and 1(c) show general information about known dialysis techniques,

FIG. 2 shows a fiber bundle according to a first preferred embodiment of the invention,

FIG. 3 shows a schematic longitudinal section of a dialyzer according to aspects of the invention in accordance with a first preferred embodiment of the invention in the assembling position,

FIG. 4 shows a schematic longitudinal section of the dialyzer according to aspects of the invention in accordance with the first preferred embodiment of the invention in a completely assembled position,

FIG. 5 shows a schematic longitudinal section of a dialyzer according to aspects of the invention in accordance with a second preferred embodiment of the invention in a completely assembled position,

FIG. 6 shows a schematic longitudinal section of a dialyzer according to aspects of the invention during assembly, and

FIG. 7 shows a schematic longitudinal section of a dialyzer according to aspects of the invention in accordance with a third preferred embodiment of the invention in a completely assembled position.

### DETAILED DESCRIPTION OF THE PREFERRED EMBODIMENTS

Hereinafter, preferred embodiments of the present invention shall be described by the example of a high-flux dialyzer including a volume-enlarging filler.

In a first embodiment, a filler capable of swelling in the presence of water, preferably a polymer capable of swelling by water, is used. Water-swelling polymers are known, for example, from DE-A-19748631. Especially preferred are water-swelling polymers in the form of homopolymers or copolymers on the basis of (meth)acrylic acid, (meth)acrylamides and/or (meth)acrylates, wherein in the copolymer any monomers adapted to be copolymerized with the afore-mentioned monomers which do not impair the swelling capability of the copolymer can be used. Preferred comonomers are acrylic nitrile, acrylate, acrylamide, allyl compounds, vinyl acetate, hydroxyethyl cellulose, hydroxypropyl cellulose, carboxymethyl cellulose, carboxypropyl cellulose and respective salts thereof (e.g. Na salts) as well as guar galactomannanum derivatives and the like. In the dry state the diameter of the bundle thus is increased by few millimeters only, thus allowing the bundle to be easily inserted into the dialyzer. Depending on where the polymer was applied, the film may remain in the dialyzer or may be removed again. The polymer ring or strip may remain in the dialyzer. When the dialyzer is flushed before the treatment, for example, the polymer soaks with the water of the flushing solution and its volume is increased. Crosslinked polyacrylic acid absorbs 500 to 1000 times its inherent weight of water. The increased volume of the filler ring counteracts the dialysate flow and on the upstream side causes an increase in pressure and on the downstream side causes a reduction of pressure. Preferably, the volume increase is set so that the blood flow in the capillaries is not influenced. In this way, on the downstream side by far more water is pressed through the capillary wall by the vacuum formed out of the blood to the dialysate side than in a conventional dialyzer. Said lacking water can be withdrawn from the dialysate by volume control of the dialysis apparatus on the upstream side of the dialyzer and can be absorbed by the blood. In this way, high internal filtration occurs inside the dialyzer without any additional apparatus, for example for controlling and/or regulating re-infusion from outside has to be added.

In the following, the structure and the manufacture of a dialyzer according to the first embodiment will be illustrated in detail by way of FIGS. 2 to 4.

FIG. 2 shows a schematic representation of a fiber bundle including a filler strip according to the first embodiment. A strip-shaped filler 20 made from dry polymer of the afore-mentioned type is applied directly to a bundle of a plurality of capillaries 10 (fiber bundle) or to a wrapping film enclosing the fiber bundle, said dry polymer having super-adsorbing properties and thus adopting a definitely increased volume after activation.

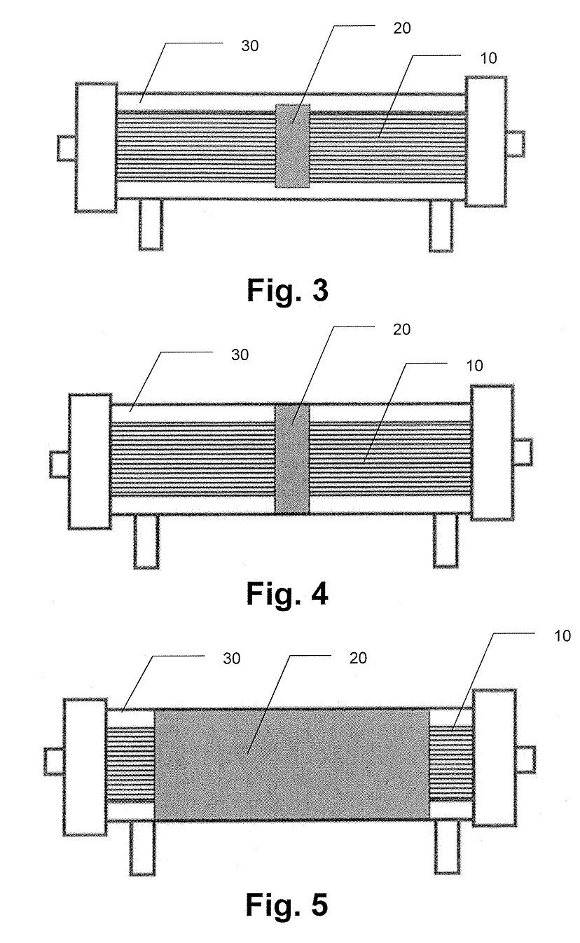

FIG. 3 shows a schematic representation of a dialyzer including a dialyzer housing 30 and an inserted fiber bundle of the capillaries 10 and the strip-shaped filler 20 according to the first embodiment. The fiber bundle is introduced (e.g. drawn) into the dialyzer housing 30. The little expansion of the dry polymer facilitates insertion.

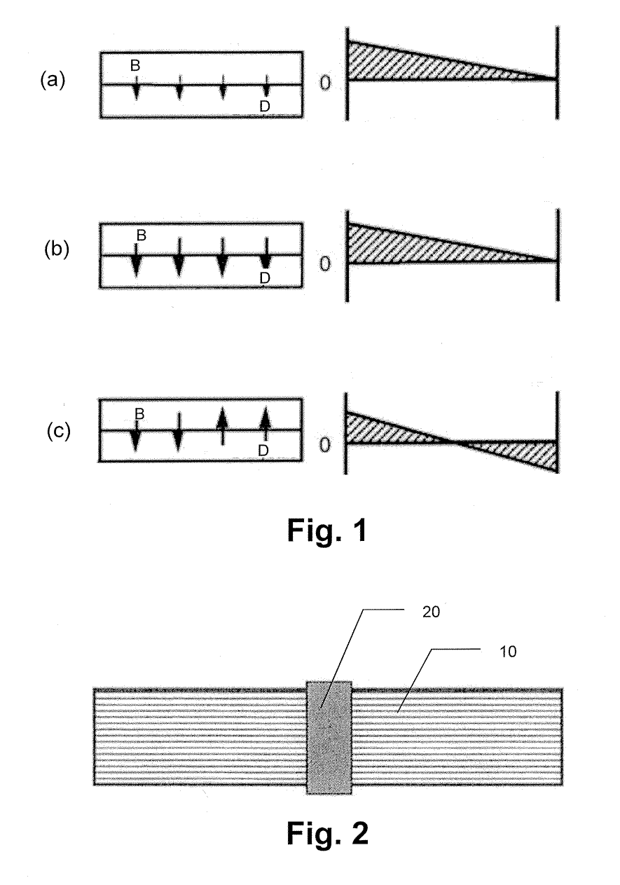

FIG. 4 shows a schematic representation of the dialyzer including the dialyzer housing 30 and the inserted fiber bundle of the capillaries 10 and the strip-shaped filler 20 after activation of the volume increase, for example by exposing the latter to water. When the fiber bundle is exposed to water, the polymer of the filler 20 absorbs water and swells. Thus, flow constriction of the dialysate is formed which then results in the pressure profile shown in FIG. 1(c) with reversed pressure gradient and improved back-filtration.

FIG. 5 shows a schematic representation of a dialyzer including the dialyzer housing 30 and the inserted fiber bundle of capillaries 10 having a strip-shaped filler 20 enlarged in the longitudinal direction of the dialyzer housing 30 according to a second embodiment after volume increase thereof. The width of the strip-shaped filler (polymer strip) 20 in this way may also extend over almost the total length of the dialyzer housing 30. This measure causes the packing density of the dialyzer to be increased, which allows an improvement of the performance data of the dialyzer to be increased as a whole.

In the afore-mentioned embodiments, the polymer of the filler 20 may be applied either packed in a water-permeable film or as a gel-type paste. WO 2003020824 A1, for example, discloses a suitable self-adhesive gel matrix on the basis of polyacrylic acid containing polyvinylpyrrolidone (PVP) as a crosslinking agent.

Furthermore, the kinetics of swelling can be adjusted by the polymer content and/or the particle size, for example.

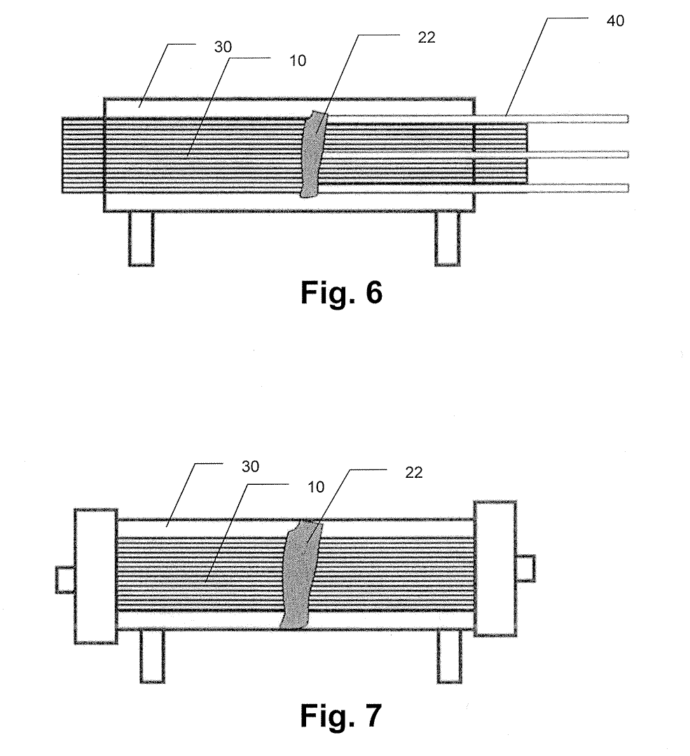

Hereinafter, an alternative third embodiment having a different configuration of the filler as polymer foam is described with reference to FIGS. 6 and 7.

FIG. 6 illustrates a schematic representation of a dialyzer with the dialyzer housing 30 being opened without any end caps including inserted nozzles 40 for introducing a foam-type filler 22 according to the third embodiment.

The polymer of the foam-type filler 22 is introduced or injected into the desired area of the dialyzer housing 30 via the long nozzles 40. By the chemical reaction during hardening a gas is formed which causes the polymer to take a foam shape and thus effectuates an increase in volume. The plastic foam system may be, for example, any one of the common foam systems used in medical engineering including e.g. a two-pack polyurethane foam, a two-pack polyurethane aerosol dosing foam and/or a two-pack epoxy resin foam. Alternatively, also silicone foam systems may be used or a polymer capable of swelling according to the first two embodiments can be introduced to a foam.

FIG. 7 shows a schematic representation of the dialyzer housing 30 including the introduced foam-type filler 22 in accordance with the third embodiment after increase in volume.

Summing up, a dialyzer and a method of manufacture thereof have been described, wherein the dialyzer includes a tubular dialyzer housing in the interior of which a plurality of capillaries 10 each extending in the longitudinal direction of the dialyzer housing 30 and being juxtaposed transversely to the longitudinal direction is arranged, with a filler 20, 22 having a volume-increasing property being arranged between the inner wall of the dialyzer housing 30 and the capillaries 10.

1.-12. (canceled)

13. A dialyzer comprising:
a tubular dialyzer housing having an interior;
a plurality of capillaries arranged within the interior of the tubular dialyzer housing, each of the plurality of capillaries extending in a longitudinal direction of the tubular dialyzer housing and being juxtaposed transversely to the longitudinal direction; and
a filler having a volume-increasing property arranged between an inner wall of the tubular dialyzer housing and the plurality of capillaries, wherein the filler is surrounded by a water-permeable film or the filler is a gel-type paste or a polymer foam configured to be injected into the tubular dialyzer housing.

14. The dialyzer according to claim 13, wherein the capillaries are wrapped with a film and the filler is arranged between the film and the inner wall of the tubular dialyzer housing.

15. The dialyzer according to claim 13, wherein the filler is configured such that an increase in volume of the filler does not influence the flow rate of the plurality of capillaries when the dialyzer is in use.

16. The dialyzer according to claim 13, wherein the filler is configured to swell by exposure to water.

17. The dialyzer according to claim 16, wherein the filler is a polymer configured swell by exposure to water.

18. The dialyzer according to claim 17, wherein the filler is a homopolymer or a copolymer based on at least one of acrylic acid, methacrylic acid, acrylamide, methacrylamide, acrylate, or methacrylate.

19. The dialyzer according to claim 13, wherein the filler is strip-shaped and is wound around the plurality of capillaries transverse to the longitudinal direction.

20. The dialyzer according to claim 19, wherein the strip-shaped filler extends in the longitudinal direction over the total length of the tubular dialyzer housing.

21. The dialyzer according to claim 20, wherein the polymer foam is a two-pack polymer foam.

22. A method of manufacturing a dialyzer comprising the steps of:
applying a filler, which is surrounded by a water-permeable film or which is a gel-type paste, having a volume-increasing characteristic to a bundle of capillaries,
introducing the bundle of capillaries including the applied filler to a dialyzer housing, and
activating a volume-increasing mechanism of the applied filler to increase the volume-increasing characteristic.

23. The method according to claim 22, wherein the volume-increasing mechanism is activated by supplying a liquid.

24. A method of manufacturing a dialyzer comprising the steps of:
introducing a bundle of capillaries into a dialyzer housing, and
activating a volume-increasing mechanism of a foam-type filler having a volume-increasing property by injecting the filler into the dialyzer housing.
